(19) United States
(12) Patent Application Publication
(10) Pub. No.: US 2022/0378809 A1
(43) Pub. Date: Dec. 1, 2022

(54) SYNTHETIC COMPOSITION FOR BALANCING THE BILE ACID PROFILE IN THE INTESTINE

(71) Applicant: GLYCOM A/S, Hørsholm (DK)

(72) Inventors: Louise Kristine VIGSNÆS, Copenhagen NV (DK); Bruce MCCONNELL, La Tour de Peilz (CH)

(21) Appl. No.: 17/755,956

(22) PCT Filed: Nov. 13, 2020
(86) PCT No.: PCT/IB2020/060692

§ 371 Date: May 12, 2022

(30) Foreign Application Priority Data
Nov. 14, 2019 (DK) PA 2019 01337

(51) Int. Cl. A61K 31/702; A61K 35/745; A61K 9/48; A61K 9/16; A61P 1/12

(52) U.S. Cl. CPC A61K 31/702; A61K 35/745; A61K 9/4825; A61K 9/1611; A61P 1/12

(57) ABSTRACT

This invention relates to a method and composition for balancing the bile acid profile in the intestine of humans, particularly decreasing primary bile acids and/or increasing production of secondary bile acids.

### FIELD OF THE INVENTION

This invention relates to a method and composition for balancing the bile acid profile in the intestine of humans, particularly decreasing primary bile acids and/or increasing production of secondary bile acids.

### BACKGROUND OF THE INVENTION

Bile acids are sterol acids synthesized in the liver which play a major role in the digestion and absorption of dietary lipids and fat-soluble vitamins in the intestine. They also significantly affect gastrointestinal motor function, sensory and secretory functions, intestinal barrier permeability and the regulation of the inflammatory responses. They also possess host signalling functions. Bile acid functioning is therefore important for gastrointestinal health and overall health. Dysfunctions in bile acid synthesis and metabolism is associated with many diseases such as liver diseases, inflammatory bowel diseases, irritable bowel syndrome, antibiotic-associated conditions, metabolic diseases such as obesity, and even cardiovascular diseases and respiratory diseases.

Bile acids are produced from cholesterol in the liver as primary bile acids, generally chenodeoxycholic acid (CDCA) and cholic acid (CA). Thereafter the primary bile acids are conjugated to either glycine or taurine to generate the conjugated primary bile acids glyco- or tauro-chenodeoxycholic acid (GCDCA/TCDCA and glycol- or tauro-cholic acid GCA/TCA). These conjugated bile acids are transferred across the canalicular membrane and carried in bile the gallbladder and stored until needed. When needed after intake of food, they are released into the duodenum. Once released into the intestine, they perform their digestive function and begin to be metabolised by the intestinal microbiota. Most bile acids are actively absorbed by specific bile acid receptors in the terminal ileum and recycled back to the liver via the portal system. A minor amount of bile acids escape absorption and enter the colon where they undergo microbial biotransformation to form the secondary bile acids. An initial step in biotransformation is deconjugation which begins in the small intestine. Deconjugation is due to bile salt hydrolase (BSH) which is produced by gut bacteria including members of lactobacilli, bifidobacteria, Clostridium and Bacteroides. Deconjugated bile acids can be metabolized through 7-dehydroxylation into secondary bile acids such as deoxycholic acid (DCA) and lithocholic acid (LCA). Bacteria with capability to produce secondary bile acids have been identified in Lachnospiraceae (clusters XIVa) and in Eubacterium, both bacterial taxa belonging to Firmicutes (Wahlström et al. Cell metabolism 24, 41 (2016)). Failure to deconjugate the primary bile acids, and failure to metabolise into secondary bile acids, leads to elevated concentrations of primary bile acids in the colon. Elevated levels of primary bile acids are linked to the diseases and conditions mentioned above.

The intestinal microbiota has the capability to alter the bile acid composition in the host by metabolising the bile acids. The intestinal microbiota is a diverse community of approximately 10¹⁴ bacterial cells comprising 500 to 1000 distinct bacterial species. The gut microbiota contains at least 100 times as many genes as the human genome, most of which confer physiological functions. These recognized roles include metabolic functions such as vitamin synthesis, regulating the uptake and deposition of dietary lipids, absorbing indigestible carbohydrates, and modulating the intestinal epithelium's absorptive capacity for optimum nutrient metabolism. Protective functions include the maintenance of intestinal barrier integrity. Due to the numerous functions of the gut microbiota important to preserve human health, recent research has been able to link imbalances in the gut bacterial population to both intra- and extraintestinal diseases. Pathological imbalances in the gut microbiota have been linked to the dysmetabolism of bile acids in the gut.

Bile acids also have the potential to alter the intestinal microbiota and immune response. Both primary and secondary bile acids can signal through two receptors, the farnesoid X receptor (FXR) and the plasma membrane-bound G protein coupled receptor (TGRS). Primary bile acids are preferential ligands for the farnesoid X receptor (FXR), while secondary bile acids are ligands for TGRS. Activation of FXR protects against bacterial overgrowth and translocation in the distal small intestine, and induces transcription of antimicrobial agents (e.g., iNOS and IL-18). TGRS can minimize production of proinflammatory cytokines (IL-1α, IL-2β, IL-6, and TNFα) stimulated by lipopolysaccharides in macrophages and Kupffer cells through inhibition of NF-kB.

Due to the impact the gut microbiota can have on bile acids, dysbiosis can result in abnormal bile acid modification resulting in the development of intra- and extraintestinal diseases. For example, in metabolic diseases, studies using germ-free and antibiotic treated mice have shown that the absence of bacteria lead to a bile acid pool consisting mainly of primary conjugated bile acids, and this can induce diet-induced obesity through farnesoid X receptor (FXR) signalling (Fiorucci et al, Trends Mol. Med. 21, 702 (2015)). In mice fed a high fat diet, the integrity of the intestinal mucosal barrier is impaired after modification of the bile acid profile with a decrease in the proportion of secondary bile acids. In liver diseases, the ratio of secondary/primary bile acids was lower in cirrhotic patients than controls. Secondary bile acids were detectable in all controls but in a significantly lower proportion in cirrhotic patients. In addition, the imbalance of the bile acid pool was linked to the abundance of key gut microbiota taxa (Kakiyama et al, J. Hepatol. 58, 949 (2013)). In intestinal bowel disease patients, the conversion of primary bile acids to secondary bile acids is impaired, and there is a significant increase of E. coli and a significant decrease of bifidobacteria and Clostrium groups involved in bile acid transformation. Hence, the altered bile acid profile in the IBD patients could lead to inflammation in IBD (Duboc et al, Neurogastroenterol. Motil. 24, 513 (2012)). Bile acid imbalance is also associated with the consequence of chronic antibiotic use. Following antibiotics, alterations in gut microbial composition and a subsequent alteration in the bile acid metabolome result in a loss of colonization resistance against C. difficile. In recurrent C. difficile patients, higher concentrations of primary bile acids have been found, while the secondary bile acids were nearly undetectable (Weingarden et al, Am. J. Physiol. Gastrointest. Liver Physiol. 306, G310 (2014)).

An imbalance in the bile acid profile has also been observed in IBS patients. For example, diarrhoea predominant IBS (IBS-D) patients have a significant increase in primary bile acids and a corresponding decrease in secondary bile acids compared to healthy controls. This correlated with a higher stool frequency and a lower stool consistency as measured by the Bristol stool chart. In addition, dysbiosis was also observed with an increase in Escherichia coli and a decrease in Clostridium leptum and Bifidobacterium (Dior et al, Neurogastroenterol. Motil. 28, 1330 (2016)). Also, FXR expression is elevated in the terminal ileum of IBS patients, and stimulating intestinal cells with CDCA increased the permeability and the release of proinflammatory cytokines. This suggests that imbalance in the bile acid profile can be involved in disruption of intestinal barrier function and cause low-grade inflammation of the small intestinal mucosa in IBS (Horikawa et al, Digestion 100, 286 (2019)).

Imbalances in bile acid profile can be treated using bile acid binders, by administering chemically synthesized bile acids, and diet. Bile acid binders such as Colestyramine, Colestipol and Colesevelam are used to sequestrate bile acids and allow them to be removed from the intestinal tract in faeces. They are commonly used to treat chronic diarrhoea. However, they treat symptoms and do not address underlying causes. Further, like all drugs, they have side effects. The oral administration of pure, chemically synthesized, secondary bile acids such as Ursodeoxycholic acid (UDCA) is used for patients with cholesterol gallstones. UDCA has also been approved to improve liver function in patients with primary biliary cirrhosis or sclerosing cholangitis, (Kim et al, Scientific reports 8:11874 (2018)). In a single case report, daily UDCA administration has also shown to successfully eliminate and prevent recurrence of C. difficile ileal pouchitis (Weingarden et al. 2015, J Clin Gastroenterol). In an animal study, daily oral administration of UDCA, tauroursodeoxycholic acid (TUDCA), or glycoursodeoxycholic acid (GUDCA) equally lowered the severity of dextran sodium sulphate-induced colitis in mice (Van den Bossche et al, Appl. Environ. Microbiol. 83, e02766 (2017)). Hence, synthesized secondary bile acids could be used as treatment in certain diseases with imbalance in the bile acid profile.

However, they also treat symptoms and do not address underlying causes and it is not clear which mixtures of secondary bile acids would be best for any patient. Also, there are side effects including increased risk of serious side effects. Diet is a safe option but it is extremely difficult for patients to manage their diet without frequent professional assistance.

Therefore, there is a need for safe, effective interventions which improve bile acid profiles in human by addressing bacterial metabolism of bile acids.

### SUMMARY OF THE INVENTION

A first aspect of this invention relates to one or more human milk oligosaccharides (HMOs) for use in decreasing primary bile acids and/or increasing production of secondary bile acids in the gastrointestinal tract of a human.

A second aspect of the invention is a synthetic composition for use in decreasing primary bile acids and/or increasing production of secondary bile acids in the gastrointestinal tract of a human, the synthetic composition comprising one or more human milk oligosaccharides (HMOs).

The synthetic composition can be a nutritional or pharmaceutical composition.

Preferably, the synthetic composition contains an amount of 0.5 g to 15 g of the one or more human milk oligosaccharides; more preferably 1 g to 10 g. For example, the synthetic composition may contain 2 g to 7.5 g of the one or more human milk oligosaccharides.

The synthetic composition may contain a bifidobacteria; for example, Bifidobacterium longum, Bifidobacterium infantis and/or Bifidobacterium bifidum.

A third aspect of the invention is a pack for use in decreasing primary bile acids and/or increasing production of secondary bile acids in the gastrointestinal tract of a human, the pack comprising at least 14 individual daily doses of an effective amount of one or more human milk oligosaccha rides.

The individual daily doses in the pack preferably contain an amount of 0.5 g to 15 g of the one or more human milk oligosaccharides; more preferably 1 g to 10 g. For example, the pack may contain 2 g to 7.5 g of the one or more human milk oligosaccharides. Further the pack preferably comprises at least about 21 individual daily doses; for example, about 28 daily doses.

Preferably, the one or more human milk oligosaccharides are selected from neutral human milk oligosaccharides. Preferably, the one or more neutral human milk oligosaccharides are selected from a fucosylated neutral human milk oligosaccharide, such as 2′-FL, 3-FL, DFL or LNFP-I, a non-fucosylated neutral human milk oligosaccharide, such as LNnT or LNT, or a mixture of both.

Preferably, the human suffers from one or more of a liver disease, an inflammatory bowel disease, a metabolic disorder, irritable bowel syndrome, and a condition associated with antibiotic treatment.

A fourth aspect of this invention is a method decreasing primary bile acids and/or increasing production of secondary bile acids in the gastrointestinal tract of a human, the method comprising orally or enterally administering to the human an effective amount of a human milk oligosaccharide.

Preferably, the decrease of primary bile acids and/or the increased production of secondary bile acids occurs in the colon of the human.

The human can be at risk of or suffer from a liver disease. For example, the liver disease can be cholesterol gallstones, cirrhosis, non-alcoholic steatohepatitis (NASH), non-alcoholic fatty liver disease (NAFLD) and/or sclerosing cholangitis.

The human can be at risk of or suffer from an inflammatory bowel disease. For example, the inflammatory bowel disease can be Crohn's disease or ulcerative colitis. Preferably, the human milk oligosaccharide is administered during a flare of the inflammatory bowel disease, during remission, or both. Preferably, the amount of the human milk oligosaccharide is sufficient to reduce sulphated bile acids.

The human can be at risk of or suffer from a metabolic disorder. For example, the metabolic disorder can be obesity, type II diabetes or syndrome X. Preferably, the amount of the human milk oligosaccharide is sufficient to decrease primary bile acids.

The human can be at risk of or suffer from irritable bowel syndrome (IBS). For example, the human can be at risk of suffer from diarrhoea predominant IBS (IBS-D), constipation predominant IBS (IBC-C) or mixed IBS (IBS-M). Preferably, in IBS-D patients, the amount of the human milk oligosaccharide is effective to decrease primary bile acids and increase production of secondary bile acids. Preferably, in IBS-C patients, the mount of the human milk oligosaccharide is effective to decrease primary bile acids.

The human can be at risk of or suffer from a condition associated with antibiotic treatment. For example, the human can be at risk of or suffer from C. difficile infection, urinary tract infection and antibiotic associated diarrhoea.

The human can be a patient suffering from C. difficile infection and the patient is administered an effective amount of a fucosylated human milk oligosaccharide to increase the concentration of deoxycholic acid (DCA) in the intestine of the patient. Deoxycholic acid advantageously inhibits outgrowth of C. difficile. The fucosylated human milk oligosaccharide is preferably 2′-FL.

Preferably, the human milk oligosaccharide is administered for at least 14 days, more preferably at least 21 days. For example, the human milk oligosaccharide may be administered for at least 28 days.

Preferably, the one or more human milk oligosaccharides are selected from neutral human milk oligosaccharides. Preferably, the one or more neutral human milk oligosaccharides are selected from a fucosylated neutral human milk oligosaccharide, such as 2′-FL, 3-FL, DFL or LNFP-I, a non-fucosylated neutral human milk oligosaccharide, such as LNnT or LNT, or a mixture of both.

Preferably, the human is administered an amount of 0.5 g to 15 g per day of the one or more human milk oligosaccharides; more preferably 1 g to 10 g per day. For example, the human may be administered 2 g to 7.5 g per day.

The human may be administered a higher dose initially followed by a lower dose. The higher dose is preferably about 3 g to about 10 g per day (for example about 4 g to about 7.5 g per day) and the lower dose is preferably about 2 g to about 7.5 g per day (for example about 2 g to about 5 g per day).

The human may be administered a bifidobacteria in addition to the one or more human milk oligosaccharides. The bifidobacteria may be, for example, Bifidobacterium longum, Bifidobacterium infantis and/or Bifidobacterium bifidum.

The human is preferably a non-infant human.

### BRIEF DESCRIPTION OF THE FIGURES

FIG. 1 illustrates the impact of human milk oligosaccharides on primary bile acids (mM) after antibiotic administration in an in vitro intestinal system.

### DETAILED DESCRIPTION OF THE INVENTION

It has now been surprisingly found that administration of one or more human milk oligosaccharides (HMOs) to humans, balances the bile acid profile in the gastro-intestinal tract the human by decreasing primary bile acids and/or increasing production of secondary bile acids. It is believed that the human milk oligosaccharides achieve this by restoring at least partially the composition or functioning of the intestinal microbiota through preferentially promoting the growth of bile acid transforming bacteria such as bifidobacteria and Lachnospiraceae (Cluster XIVa). As an outcome, a more beneficial intestinal microbial community is obtained which shapes and maintains the intestinal environment including the bile acid profile. In particular, the decrease of primary bile acids is promoted in a first step, for example, by intestinal microbiota which produce bile salt hydrolase (BSH) to deconjugate the primary bile acids. Thereafter the deconjugated bile acids are metabolised by the intestinal microbiota through various mechanisms. The production of secondary bile acids is promoted by intestinal microbiota which, for example, promote 7α-dehydroxylation.

In this specification, the following terms have the following meanings:

“Bifidobacterium of the B. adolescentis phylogenetic group” means a bacterium selected from the group consisting of Bifidobacterium adolescentis, Bifidobacterium angulatum, Bifidobacterium catenulatum, Bifidobacterium pseudocatenulatum, Bifidobacterium kashiwanohense, Bifidobacterium dentum and Bifidobacterium stercorin (Duranti et al, Appl. Environ. Microbiol. 79, 336 (2013), Bottacini et al, Microbial. Cell Fact. 13:S4 (2014)). Preferably, a Bifidobacterium of the B. adolescentis phylogenetic group is Bifidobacterium adolescentis and/or Bifidobacterium pseudocatenulatum.

“Enteral administration” means any conventional form for delivery of a composition to a human that causes the deposition of the composition in the gastrointestinal tract (including the stomach). Methods of enteral administration include feeding through a naso-gastric tube or jejunum tube, oral, sublingual and rectal.

“Effective amount” means an amount of a composition that provides an HMO in a sufficient amount to render a desired treatment outcome in a human. An effective amount can be administered in one or more doses to achieve the desired treatment outcome.

“Human milk oligosaccharide” or “HMO” means a complex carbohydrate found in human breast milk (Urashima et al.: Milk Oligosaccharides. Nova Science Publisher (2011); Chen Adv. Carbohydr. Chem. Biochem. 72, 113 (2015)). The HMOs have a core structure comprising a lactose unit at the reducing end that can be elongated by one or more β-N-acetyl-lactosaminyl and/or one or β-more lacto-N-biosyl units, and which core structure can be substituted by an α L-fucopyranosyl and/or an α-N-acetyl-neuraminyl (sialyl) moiety. In this regard, the non-acidic (or neutral) HMOs are devoid of a sialyl residue, and the acidic HMOs have at least one sialyl residue in their structure. The non-acidic (or neutral) HMOs can be fucosylated or non-fucosylated. Examples of such neutral non-fucosylated HMOs include lacto-N-tetraose (LNT), lacto-N-neotetraose (LNnT), lacto-N-neohexaose (LNnH), para-lacto-N-neohexaose (pLNnH), para-lacto-N-hexaose (pLNH) and lacto-N-hexaose (LNH). Examples of neutral fucosylated HMOs include 2′-fucosyllactose (2′-FL), lacto-N-fucopentaose I (LNFP-I), lacto-N-difucohexaose I (LNDFH-I), 3-fucosyllactose (3-FL), difucosyllactose (DFL), lacto-N-fucopentaose II (LNFP-II), lacto-N-fucopentaose III (LNFP-III), lacto-N-difucohexaose III (LNDFH-III), fucosyl-lacto-N-hexaose II (FLNH-II), lacto-N-fucopentaose V (LNFP-V), lacto-N-difucohexaose II (LNDFH-II), fucosyl-lacto-N-hexaose I (FLNH-I), fucosyl-para-lacto-N-hexaose I (FpLNH-I), fucosyl-para-lacto-N-neohexaose II (FpLNnH II) and fucosyl-lacto-N-neohexaose (FLNnH). Examples of acidic HMOs include 3′-sialyllactose (3′-SL), 6′-sialyllactose (6′-SL), 3-fucosyl-3′-sialyllactose (FSL), LST a, fucosyl-LST a (FLST a), LST b, fucosyl-LST b (FLST b), LST c, fucosyl-LST c (FLST c), sialyl-LNH (SLNH), sialyl-lacto-N-hexaose (SLNH), sialyl-lacto-N-neohexaose I (SLNH-I), sialyl-lacto-N-neohexaose II (SLNH-II) and disialyl-lacto-N-tetraose (DSLNT).

“Irritable bowel syndrome” and “IBS” mean a group of functional bowel disorders of humans, particularly adults, characterised by one or more chronic symptoms including abdominal pain, abdominal discomfort, abdominal bloating, fatigue, and changes in bowel movement patterns, such as patterns of loose or more frequent bowel movements, diarrhoea and constipation, typically in the absence of any apparent structural abnormality. There are at least three forms of IBS, depending on which symptom predominates: (1) diarrhoea-predominant (IBS-D); (2) constipation-predominant (IBS-C); and (3) IBS with alternating stool pattern (IBS-M). There are also various clinical subtypes of IBS, such as post-infectious IBS (IBS-PI).

“Microbiota”, “microflora” and “microbiome” mean a community of living microorganisms that typically inhabits a bodily organ or part, particularly the gastro-intestinal organs of humans. The most dominant members of the gastrointestinal microbiota include microorganisms of the phyla of Firmicutes, Bacteroidetes, Actinobacteria, Proteobacteria, Synergistetes, Verrucomicrobia, Fusobacteria, and Euryarchaeota; at genus level Bacteroides, Faecalibacterium, Bifidobacterium, Roseburia, Alistipes, Collinsella, Blautia, Coprococcus, Ruminococcus, Eubacterium and Dorea; at species level Bacteroides uniformis, Alistipes putredinis, Parabacteroides merdae, Ruminococcus bromii, Dorea longicatena, Bacteroides caccae, Bacteroides thetaiotaomicron, Eubacterium hallii, Ruminococcus torques, Faecalibacterium prausnitzii, Ruminococcus lactaris, Collinsella aerofaciens, Dorea formicigenerans, Bacteroides vulgatus and Roseburia intestinalis. The gastrointestinal microbiota includes the mucosa-associated microbiota, which is located in or attached to the mucous layer covering the epithelium of the gastrointestinal tract, and luminal-associated microbiota, which is found in the lumen of the gastrointestinal tract.

“Modulating of microbiota” means exerting a modifying or controlling influence on microbiota, for example an influence leading to an increase in the indigenous intestinal abundance of Bifidobacterium, and/or butyrate producing bacteria. In another example, the influence may lead to a reduction of the intestinal abundance of Ruminococcus gnavus and/or Proteobacteria. “Proteobacteria” are a phylum of Gram-negative bacteria and include a wide variety of pathogenic bacteria, such as Escherichia, Salmonella, Vibrio, Helicobacter, Yersinia and many other notable genera.

“Non-infant human” or “non-infant” means a human of 3 years of age and older. A non-infant human can be a child, a teenager, an adult or an elderly person.

“Oral administration” means any conventional form for the delivery of a composition to a human through the mouth. Accordingly, oral administration is a form of enteral administration.

“Preventive treatment” or “prevention” means treatment given or action taken to diminish the risk of onset or recurrence of a disease.

“Relative abundance of a bacteria” means the abundance of that bacteria relative to other bacteria in the microbiota of the gastrointestinal tract of a human.

“Relative growth of a bacteria” means the growth of a bacteria relative to other bacteria in the microbiota in the gastrointestinal tract of humans.

“Secondary prevention” means prevention of onset of the condition in a high-risk patient, or prevention or reduction of reoccurrence of symptoms in a patient who has already has the condition. A “high-risk” patient is an individual who is predisposed to developing the condition; for example, a person with a family history of the condition

“Synthetic composition” means a composition which is artificially prepared and preferably means a composition containing at least one compound that is produced ex vivo chemically and/or biologically, e.g. by means of chemical reaction, enzymatic reaction or recombinantly. The synthetic composition typically comprises one or more HMOs. Also, in some embodiments, the synthetic compositions may comprise one or more nutritionally or pharmaceutically active components which do not affect adversely the efficacy of the HMOs. Some non-limiting embodiments of a synthetic composition of the invention are described below.

“Therapy” means treatment given or action taken to reduce or eliminate symptoms of a disease or pathological condition.

“Treat” means to address a medical condition or disease with the objective of improving or stabilising an outcome in the person being treated or addressing an underlying nutritional need. Treat therefore includes the dietary or nutritional management of the medical condition or disease by addressing nutritional needs of the person being treated. “Treating” and “treatment” have grammatically corresponding meanings.

The HMOs can be isolated or enriched by well-known processes from milk(s) secreted by mammals including, but not limited to human, bovine, ovine, porcine, or caprine species. The HMOs can also be produced by well-known processes using microbial fermentation, enzymatic processes, chemical synthesis, or combinations of these technologies. As examples, using chemistry LNnT can be made as described in WO 2011/100980 and WO 2013/044928, LNT can be synthesized as described in WO 2012/155916 and WO 2013/044928, a mixture of LNT and LNnT can be made as described in WO 2013/091660, 2′-FL can be made as described in WO 2010/115934 and WO 2010/115935, 3-FL can be made as described in WO 2013/139344, 6′-SL and salts thereof can be made as described in WO 2010/100979, sialylated oligosaccharides can be made as described in WO 2012/113404 and mixtures of human milk oligosaccharides can be made as described in WO 2012/113405. As examples of enzymatic production, sialylated oligosaccharides can be made as described in WO 2012/007588, fucosylated oligosaccharides can be made as described in WO 2012/127410, and advantageously diversified blends of human milk oligosaccharides can be made as described in WO 2012/156897 and WO 2012/156898. Biotechnological methods which describe how to make core (non-fucosylated neutral) human milk oligosaccharides optionally substituted by fucose or sialic acid using genetically modified E. coli con be found in WO 01/04341 and WO 2007/101862.

The HMO may be a single HMO or a mixture of any HMOs suitable for the purpose of the invention. In one embodiment, the mixture comprises neutral HMOs, preferably at least a first neutral HMO and at least a second neutral HMO. The first neutral HMO is a fucosylated neutral HMO and the second neutral HMO is a core HMO (also referred to as non-fucosylated neutral

HMO). Particularly, the mixture of HMOs may contain a fucosylated HMO selected from the list consisting of 2′-FL, 3-FL, DFL, LNFP-I, LNFP-II, LNFP-III, LNFP-V, LNDFH-I, LNDFH-II, LNDFH-III, FLNH-I, FLNH-II, FLNnH, FpLNH-I and F-pLNnH II, and a core HMO selected from the list consisting of LNT, LNnT, LNH, LNnH, pLNH and pLNnH. More preferably, the mixture of neutral HMOs contains, consists of or essentially consists of, a fucosylated HMO selected from the list consisting of 2′-FL, 3-FL, DFL and LNFP-I, and a core HMO selected from the list consisting of LNT and LNnT; advantageously the mixture comprises, consists of or essentially consists of, 2′-FL and at least one of LNnT and LNT; or at least one of 2′-FL and DFL and at least one of LNnT and LNT; or 2′-FL, DFL and at least one of LNnT and LNT.

In other embodiment, the mixture comprises at least a first (acidic) HMO and at least a second (neutral) HMO, wherein the first (acidic) HMO is selected from the list consisting of 3′-SL, 6′-SL and FSL and the second (neutral) HMO is selected from the list consisting of 2′-FL, 3-FL, DFL, LNFP-I, LNT and LNnT. Advantageously the mixture comprises 2′-FL and 6′-SL; or 6′-SL and at least one of 2′-FL and DFL; or 2′-FL, 6′-SL and at least one of LNnT and LNT; or 2′-FL, DFL, 6′-SL and at least one of LNnT and/or LNT.

The synthetic composition can be in the form of a nutritional composition. For example, the nutritional composition can be a food composition, a rehydration solution, a medical food or food for special medical purposes, a nutritional supplement and the like. The nutritional composition can contain sources of protein, lipids and/or digestible carbohydrates and can be in powdered or liquid forms. The composition can be designed to be the sole source of nutrition or as a nutritional supplement.

Suitable protein sources include milk proteins, soy protein, rice protein, pea protein and oat protein, or mixtures thereof. Milk proteins can be in the form of milk protein concentrates, milk protein isolates, whey protein or casein, or mixtures of both. The protein can be whole protein or hydrolysed protein, either partially hydrolysed or extensively hydrolysed. Hydrolysed protein offers the advantage of easier digestion which can be important for humans with inflamed or compromised GI tracts. The protein can also be provided in the form of free amino acids. The protein can comprise about 5% to about 30% of the energy of the nutritional composition, normally about 10% to 20%.

The protein source can be a source of glutamine, threonine, cysteine, serine, proline, or a combination of these amino acids. The glutamine source can be a glutamine dipeptide and/or a glutamine enriched protein. Glutamine can be included due to the use of glutamine by enterocytes as an energy source. Threonine, serine and proline are important amino acids for the production of mucin. Mucin coats the gastrointestinal tract and can improve intestinal barrier function and mucosal healing. Cysteine is a major precursor of glutathione, which is key for the antioxidant defences of the body.

Suitable digestible carbohydrates include maltodextrin, hydrolysed or modified starch or corn starch, glucose polymers, corn syrup, corn syrup solids, high fructose corn syrup, rice-derived carbohydrates, pea-derived carbohydrates, potato-derived carbohydrates, tapioca, sucrose, glucose, fructose, sucrose, lactose, honey, sugar alcohols (e.g. maltitol, erythritol, sorbitol), or mixtures thereof. Preferably, the composition is reduced in or free from added lactose or other FODMAP carbohydrates. Generally digestible carbohydrates provide about 35% to about 55% of the energy of the nutritional composition. A particularly suitable digestible carbohydrate is a low dextrose equivalent (DE) maltodextrin.

Suitable lipids include medium chain triglycerides (MCT) and long chain triglycerides (LCT). Preferably, the lipid is a mixture of MCTs and LCTs. For example, MCTs can comprise about 30% to about 70% by weight of the lipids, more specifically about 50% to about 60% by weight. MCTs offer the advantage of easier digestion which can be important for humans with inflamed or compromised GI tracts. Generally, the lipids provide about 35% to about 50% of the energy of the nutritional composition. The lipids can contain essential fatty acids (omega-3 and omega-6 fatty acids). Preferably, these polyunsaturated fatty acids provide less than about 30% of total energy of the lipid source.

Suitable sources of long chain triglycerides are rapeseed oil, sunflower seed oil, palm oil, soy oil, milk fat, corn oil, high oleic oils, and soy lecithin. Fractionated coconut oils are a suitable source of medium chain triglycerides. The lipid profile of the nutritional composition is preferably designed to have a polyunsaturated fatty acid omega-6 (n-6) to omega-3 (n-3) ratio of about 4:1 to about 10:1. For example, the n-6 to n-3 fatty acid ratio can be about 6:1 to about 9:1 (by weight).

The nutritional composition may also include vitamins and minerals. If the nutritional composition is intended to be a sole source of nutrition, it preferably includes a complete vitamin and mineral profile. Examples of vitamins include vitamins A, B-complex (such as B1, B2, B6 and B12), C, D, E and K, niacin and acid vitamins such as pantothenic acid, folic acid and biotin. Examples of minerals include calcium, iron, zinc, magnesium, iodine, copper, phosphorus, manganese, potassium, chromium, molybdenum, selenium, nickel, tin, silicon, vanadium and boron.

The nutritional composition can also include a carotenoid such as lutein, lycopene, zeaxanthin, and beta-carotene. The total amount of carotenoid included can vary from about 0.001 μg/ml to about 10 μg/ml. Lutein can be included in an amount of from about 0.001 μg/ml to about 10 μg/ml, preferably from about 0.044 μg/ml to about 5 μg/ml of lutein. Lycopene can be included in an amount from about 0.001 μg/ml to about 10 μg/ml, preferably about 0.0185 μg/ml to about 5 μg/ml of lycopene. Beta-carotene can comprise from about 0.001 μg/ml to about 10 mg/ml, for example about 0.034 μg/ml to about 5 μg/ml of beta-carotene.

The nutritional composition preferably also contains reduced concentrations of sodium; for example, from about 300 mg/l to about 400 mg/l. The remaining electrolytes can be present in concentrations set to meet needs without providing an undue renal solute burden on kidney function. For example, potassium is preferably present in a range of about 1180 to about 1300 mg/l; and chloride is preferably present in a range of about 680 to about 800 mg/l.

The nutritional composition can also contain various other conventional ingredients such as preservatives, emulsifying agents, thickening agents, buffers, fibres and prebiotics (e.g. fructooligosaccha rides, galactooligosaccharides), probiotics (e.g. B. animalis subsp. lactis BB-12, B. lactis HN019, B. lactis Bi07, B. infantis ATCC 15697, L. rhamnosus GG, L. rhamnosus HNOOI, L. acidophilus LA-5, L. acidophilus NCFM, L. fermentum CECT5716, B. longum BB536, B. longum AH1205, B. longum AH1206, B. breve M-16V, L. reuteri ATCC 55730, L. reuteri ATCC PTA-6485, L. reuteri DSM 17938), antioxidant/anti-inflammatory compounds including tocopherols, carotenoids, ascorbate/vitamin C, ascorbyl palmitate, polyphenols, glutathione, and superoxide dismutase (melon), other bioactive factors (e.g. growth hormones, cytokines, TFG-β), colorants, flavours, and stabilisers, lubricants, and so forth.

The nutritional composition can be formulated as a soluble powder, a liquid concentrate, or a ready-to-use formulation. The composition can be fed to a human in need via a nasogastric tube or orally. Various flavours, fibres and other additives can also be present.

The nutritional compositions can be prepared by any commonly used manufacturing techniques for preparing nutritional compositions in solid or liquid form. For example, the composition can be prepared by combining various feed solutions. A protein-in-fat feed solution can be prepared by heating and mixing the lipid source and then adding an emulsifier (e.g. lecithin), fat soluble vitamins, and at least a portion of the protein source while heating and stirring. A carbohydrate feed solution is then prepared by adding minerals, trace and ultra-trace minerals, thickening or suspending agents to water while heating and stirring. The resulting solution is held for 10 minutes with continued heat and agitation before adding carbohydrates (e.g. the HMOs and digestible carbohydrate sources). The resulting feed solutions are then blended together while heating and agitating and the pH adjusted to 6.6-7.0, after which the composition is subjected to high-temperature short-time processing during which the composition is heat treated, emulsified and homogenized, and then allowed to cool. Water soluble vitamins and ascorbic acid are added, the pH is adjusted to the desired range if necessary, flavours are added, and water is added to achieve the desired total solid level.

For a liquid product, the resulting solution can then be aseptically packed to form an aseptically packaged nutritional composition. In this form, the nutritional composition can be in ready-to-feed or concentrated liquid form. Alternatively, the composition can be spray-dried and processed and packaged as a reconstitutable powder.

When the nutritional product is a ready-to-feed nutritional liquid, it may be preferred that the total concentration of HMOs in the liquid, by weight of the liquid, is from about 0.1% to about 1.5%, including from about 0.2% to about 1.0%, for example from about 0.3% to about 0.7%. When the nutritional product is a concentrated nutritional liquid, it may be preferred that the total concentration of HMOs in the liquid, by weight of the liquid, is from about 0.2% to about 3.0%, including from about 0.4% to about 2.0%, for example from about 0.6% to about 1.5%.

In another embodiment, the nutritional composition is in a unit dosage form. The unit dosage form can contain an acceptable food-grade carrier, e.g. phosphate buffered saline solution, mixtures of ethanol in water, water and emulsions such as an oil/water or water/oil emulsion, as well as various wetting agents or excipients. The unit dosage form can also contain other materials that do not produce an adverse, allergic or otherwise unwanted reaction when administered to a human. The carriers and other materials can include solvents, dispersants, coatings, absorption promoting agents, controlled release agents, and one or more inert excipients, such as starches, granulating agents, microcrystalline cellulose, diluents, lubricants, binders, and disintegrating agents. Preferably, carriers and other materials are low in FODMAPs or contain no FODMAPs.

A unit dosage form of this invention can be administered orally, e.g. as a tablet, capsule, or pellet containing a predetermined amount of the mixture, or as a powder or granules containing a predetermined concentration of the mixture or a gel, paste, solution, suspension, emulsion, syrup, bolus, electuary, or slurry, in an aqueous or non-aqueous liquid, containing a predetermined concentration of the mixture. An orally administered composition can include one or more binders, lubricants, inert diluents, flavouring agents, and humectants. An orally administered composition such as a tablet can optionally be coated and can be formulated to provide sustained, delayed or controlled release of the HMO.

A unit dosage form of this invention can also be administered by naso-gastric tube or direct infusion into the GI tract or stomach.

A unit dosage form of this invention can also include therapeutic agents such as antibiotics, probiotics, analgesics, and anti-inflammatory agents. The proper dosage of such a composition for a human can be determined in a conventional manner, based upon factors such as the human's condition, immune status, body weight and age. In some cases, the dosage will be at a concentration similar to that found for the HMOs of the composition in human breast milk. The required amount would generally be in the range from about 0.5 g to about 15 g per day, in certain embodiments from about 1 g to about 10 g per day, for example about 2 g to about 7.5 g per day. Appropriate dose regimes can be determined by methods known to those skilled in the art.

In further embodiment, the HMO can be formulated as a pharmaceutical composition. The pharmaceutical composition can contain a pharmaceutically acceptable carrier, e.g. phosphate buffered saline solution, mixtures of ethanol in water, water and emulsions such as an oil/water or water/oil emulsion, as well as various wetting agents or excipients. The pharmaceutical composition can also contain other materials that do not produce an adverse, allergic or otherwise unwanted reaction when administered to humans. The carriers and other materials can include solvents, dispersants, coatings, absorption promoting agents, controlled release agents, and one or more inert excipients, such as starches, granulating agents, microcrystalline cellulose, diluents, lubricants, binders, and disintegrating agents. Preferably, carriers and other materials are low in FODMAPs or contain no FODMAPs.

The pharmaceutical compositions can be administered orally, e.g. as a tablet, capsule, or pellet containing a predetermined amount, or as a powder or granules containing a predetermined concentration or a gel, paste, solution, suspension, emulsion, syrup, bolus, electuary, or slurry, in an aqueous or non-aqueous liquid, containing a predetermined concentration. Orally administered compositions can include binders, lubricants, inert diluents, flavouring agents, and humectants. Orally administered compositions such as tablets can optionally be coated and can be formulated to provide sustained, delayed or controlled release of the mixture therein.

The pharmaceutical compositions can also be administered by rectal suppository, aerosol tube, naso-gastric tube or direct infusion into the GI tract or stomach.

The pharmaceutical compositions can also include therapeutic agents such as antibiotics, probiotics, analgesics, and anti-inflammatory agents. The proper dosage of these compositions for a human can be determined in a conventional manner, based upon factors such condition, immune status, body weight and age. In some cases, the dosage will be at a concentration similar to that found for the HMOs in human breast milk. The required amount would generally be in the range from about 0.5 g to about 15 g per day, in certain embodiments from about 1 g to about 10 g per day, for example from about 2 g to about 7.5 g per day. Appropriate dose regimes can be determined by conventional methods.

The amount of HMOs required to be administered for decreasing primary bile acids and/or increasing production of secondary bile acids in the gastrointestinal tract of a human, will vary depending upon factors such as the risk and severity of the underlying condition, any other medical conditions or diseases, age, the form of the composition, and other medications being administered. Further the amount may vary depending upon whether the HMOs are being used to deliver a direct effect (when the dose may be higher) or whether the HMOs are being used as a secondary prevention/maintenance (when the dose may be lower). However, the required amount can be readily set by a medical practitioner and would generally be in the range from about 0.5 g to about 15 g per day, in certain embodiments from about 1 g to about 10 g per day, for example from about 2 g to about 7.5 g per day. An appropriate dose can be determined based on several factors, including, for example, body weight and/or condition, the severity of the underlying condition being treated or prevented, other ailments and/or diseases, the incidence and/or severity of side effects and the manner of administration. Appropriate dose ranges may be determined by methods known to those skilled in the art. During an initial treatment phase, the dosing can be higher (for example 3 g to 15 g per day, preferably 4 g to 7.5 g per day). During a maintenance phase, the dosing can be reduced (for example, 1 g to 10 g per day, preferably 2 g to 7.5 g per day, more preferably about 2 g to about 5 g per day)).

### EXAMPLES

The working example described herein are for illustration purposes only and should not be considered as limiting.

### Example 1—In Vitro Intestine Model

An in vitro intestinal system is used to simulate the colon region in a human infected with C. difficile. The system is inoculated with fresh faecal samples from a healthy individual aged >65 years. The system is run for two weeks as a set up period to stabilise the system. The system is fed daily with a feed solution and with bile acids (primarily taurocholic acid and glycocholic acid). Afterwards, two different interventions are run in parallel for 4 weeks.
Intervention: daily addition of 2′-FL and LNnT (ratio 4:1 by weight) plus an antibiotic (Vancomycin) for seven days, followed by 2′-FL and LNnT (ratio 4:1 by weight) without antibiotic for the next 3 weeks (wash out period),
Control: an antibiotic (Vancomycin) for seven days, followed by no intervention for the next 3 weeks (wash out period).

During the antibiotic treatment and washout periods, both the intervention and the control receive the same daily feed including bile acids. At three time points (end of set up period, end of antibiotic treatment, and end of wash out period), the microbiota community and bile acids are measured using 16S sequencing and HPLC-UV method, respectively.

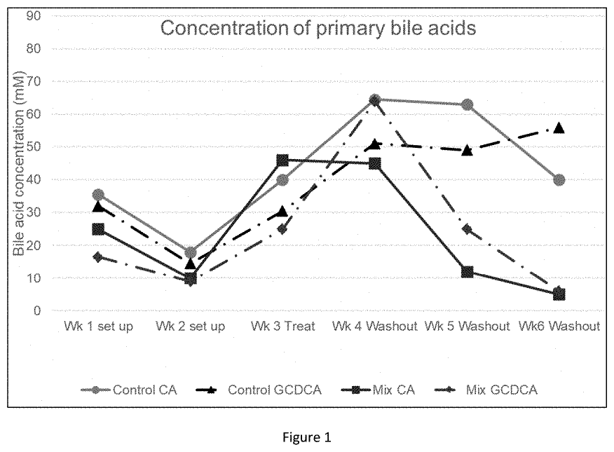

In both the intervention and control, the antibiotic treatment results in a substantial dysbiosis in the colonic microbiota. This in turn results in an impaired bile acid metabolism and a bile acid profile having high concentrations of primary bile acids. At the end of the washout period, the microbiota of the intervention system is restored, and the colonic bile acid metabolism is re-established. As shown in FIG. 1, a decrease in primary bile acids (cholic acid (CA) and glyco-chenodeoxycholic acid(GCDCA) occurs in the intervention system. For the control group, the restoration of the microbiota is incomplete and primary bile acid concentrations remain high.

### Example 2—In Vitro Intestine Model

The in vitro intestinal system is run as in example 1 except that the intervention is a daily addition of 2′-FL plus an antibiotic (Vancomycin) for seven days, followed by 2′-FL alone for the next 3 weeks (wash out period). The control is as in example b 1.
Control: an antibiotic (Vancomycin) for seven days, followed by no intervention for the next 3 weeks (wash out period).

In both the intervention and control, the antibiotic treatment results in a substantial dysbiosis in the colonic microbiota. This in turn results in an impaired bile acid metabolism and a bile acid profile having high concentrations of primary bile acids. At the end of the washout period, the microbiota of the intervention system is restored, and the colonic bile acid metabolism is re-established. As in example 1, a decrease in primary bile acids (cholic acid (CA) and glyco-chenodeoxycholic acid (GCDCA) occurs in the intervention system. Further, measure of secondary bile acids indicates the presence of the secondary bile acid deoxycholic acid (DCA).

For the control group, the restoration of the microbiota is incomplete, primary bile acid concentrations remain high and no secondary bile acids are identified.

### Example 3—Human Trial

A total of 60 male and female IBS patients are recruited to participate in the study. After a screening visit and run-in period of 1-2 weeks, the patients are selected. The patients are randomised into three groups, each of 20 patients, with two groups consuming the treatment product and one group the placebo product. The treatment groups receive either 5 grams of a combination of 2′-FL and LNnT in a 4:1 ratio by weight, or 10 grams of a combination of 2′-FL and LNnT in a 4:1 ratio by weight. The placebo group receives 5 grams glucose. Both products are in powder form in a unit dosage container.

The patients are eligible to participate if they are at an age between 18-60 years, fulfil definition of IBS-D, IBS-C or IBS-M according to the Rome IV criteria for IBS and have a global IBS-SSS score of >174 during the 2 weeks run-in period. All recruited patients are able and willing to understand and comply with the study procedures. Patients are excluded if: they have any known gastrointestinal disease(s) that may cause symptoms or interfere with the trial outcome, in particular lactose intolerance and coeliac disease; they have participated in a clinical study one month prior to screening visit; they have abnormal results in the screening tests which are clinically relevant for study participation; they are suffering for a severe disease such as malignancy, diabetes, severe coronary disease, kidney disease, neurological disease, or severe psychiatric disease or any condition which can confound the results of the study; used highly dosed probiotic supplements (yoghurt allowed) for 1 months prior to the study; consumed antibiotic drugs 1 months prior to the study; consumed on a regular basis any medication that might interfere with symptom evaluation 2 weeks prior to the study; diagnosed with and treated for IBS for more than 10 years; and pregnant or lactating.

At the screening visit (visit 1), clinical and medical history and concomitant medication is registered. IBS diagnostic criteria will be assessed and part 2 of the IBS-SSS questionnaire is completed.

A faecal sample kit is distributed together with a Bristol Stool Form Scale (BSFS) and Bowel Movement Diary (BMD) which is to be filled in during the 7 days just prior to the second visit. Patients are asked to register their diet 3 days just prior to visit 2 and are reminded not to change their usual diet during the study.

At the second visit (visit 2), eligibility criteria are checked, and eligible subjects are randomised to the three arms in the trial. A physical examination is done and several questionnaires (GSRS-IBS, IBS-SSS, HADS, NRS-11, VSI, IBS-QOL and PHQ-15 scales) are answered. Questionnaires are filled in electronically. Those who are unable or unwilling to use the electronic system fill out the questionnaires on paper. Based on clinical symptoms and data from questionnaires, patients are characterised into one of the three following groups; diarrhoea predominant (IBS-D), constipation predominant (IBS-C) or mixed (IBS-M). This enables allocation of patients from each subgroup into the intervention groups. Patients are asked about any adverse events and any changes in their usual medication. The BSFS and BMD are collected and new forms, to be filled in daily during the intervention period, are distributed. Faecal samples are collected and equipment for new samples are distributed. Blood samples are collected for routine clinical chemistry and haematology and biomarker analysis and a saliva sample is collected to analyse FUT2 secretor status. Diet records are collected, and new forms are distributed. The randomised patients are then given a 4-week supply of the placebo product or one of the treatment products depending upon the group they are randomised to. The patients and clinical staff are blinded to which product is received. Patients are instructed to consume the intervention products in the morning with breakfast.

At the third visit (visit 3) after 4 weeks, a physical examination is performed and a number of questionnaires (GSRS-IBS, IBS-SSS, HADS, NRS-11, VSI, IBS-QOL and PHQ-15 scales) are answered. Questionnaires are filled in electronically. Those who are unable or unwilling to use the electronic system fill out the questionnaires on paper. Faecal samples are collected, the BSFS and BMD are collected, and food and compliance diaries are collected to check compliance. Blood samples are collected for routine clinical chemistry and haematology and biomarker analysis. Patients are asked about any adverse events and any changes in their usual medication.

To assess the microbiota profile, DNA is extracted from faecal samples using a 96-well PowerSoil DNA Isolation Kit (MO-BIO). A minimum of one sample-well per plate is kept empty to serve as a negative control during PCR. PCR is done with the forward primer S-D-Bact-0341-b-S-17 and reverse primer S-D-Bact-0785-a-A-21 with Illumina adapters attached (Klindworth et al. Nucleic Acids Res. 41, el (2013)). These are universal bacterial 16S rDNA primers, which target the V3-V4 region. Following PCR program is used: 98° C. for 30 sec, 25× (98° C. for 10 s, 55° C. for 20 s, 72° C. for 20 s), 72° C. for 5 min. Amplification is verified by running the products on a 1% agarose gel. Barcodes are added in a nested PCR using the Nextera Index Kit V2 (Illumina) with the following PCR program: 98° C. for 30 sec, 8× (98° C. for 10 s, 55° C. for 20 s, 72° C. for 20 s), 72° C. for 5 min. Attachment of primers is verified by running the products on a 1% agarose gel. Products from the nested PCR are normalized using the SequalPrep Normalization Plate Kit and pooled. Pooled libraries are concentrated by evaporation and the DNA concentration of pooled libraries is measured on a Qubit fluorometer using the Qubit High Sensitivity Assay Kit (Thermo Fisher Scientific). Sequencing is done on a MiSeq desktop sequencer using the MiSeq Reagent Kit V3 (Illumina) for 2×300 bp paired-end sequencing. The 64-bit version of USEARCH is used for bioinformatical analysis of the sequence data.

Between Visit 2 and Visit 3, all patients tolerate the interventions with no difference in tolerance between the groups. All patients improve gastrointestinal symptoms. Patients receiving the treatment products have elevated bifidobacteria levels as compared to the placebo group at Visit 3. Further patients receiving the treatment products have reduced concentrations of primary bile acids and increased concentrations of secondary bile acids in faeces.

### Example 4—Capsule Composition

A capsule is prepared by filling about 1 g of HMO into a 000 gelatine capsule using a filing machine. The capsules are then closed. The HMO are in free flowing, powder form.

### Example 5—Nutritional Composition

The HMOs 2′-FL and LNnT are introduced into a rotary blender in a 4:1 mass ratio. An amount of 0.25 w % of silicon dioxide is introduced into the blender and the mixture blended for 10 minutes. The mixture is then agglomerated in a fluidised bed and filled into 5 gram stick packs and the packs are sealed.

1.-9. (canceled)

10. A method for decreasing primary bile acids and/or increasing production of secondary bile acids in the gastrointestinal tract of a human, the method comprising orally or enterally administering to the human an effective amount of a synthetic composition consisting essentially of one or more human milk oligosaccharides (HMOs) and optionally bifidobacteria, wherein the synthetic composition is artificially prepared and contains at least one compound that is produced ex vivo chemically and/or biologically.

11. The method of claim 10, wherein the gastrointestinal tract is the colon.

12. The method of claim 10, wherein the human is at risk of or suffers from a liver disease or condition.

13. The method of claim 10, wherein the human is at risk of or suffers from an inflammatory bowel disease.

14. The method of claim 13, wherein the one or more human milk oligosaccharides is administered during a flare of the inflammatory bowel disease.

15. The method of claim 10, wherein the human is at risk of or suffers from a metabolic disorder.

16. The method of claim 10, wherein the human is at risk of or suffers from irritable bowel syndrome (IBS).

17. The method of claim 16, wherein the human is at risk of suffers from diarrhoea predominant IBS (IBS-D) and the effective amount of the one or more human milk oligosaccharides is effective to decrease primary bile acids and increase production of secondary bile acids.

18. The method of claim 16, wherein the human is at risk of suffers from constipation predominant IBS (IBC-C) or mixed IBS (IBS-M) and the effective amount of the one or more human milk oligosaccharides is effective to decrease primary bile acids.

19. The method of claim 10 in which the human is at risk of or suffers from a condition associated with antibiotic treatment.

20. The method of claim 10, wherein the one or more human milk oligosaccharides is one or more neutral human milk oligosaccharides; one or more fucosylated neutral human milk oligosaccharides; one or more non-fucosylated neutral human milk oligosaccharides; or a mixture of both.

21. A method of increasing the concentration of deoxycholic acid (DCA) in the intestine of a patient suffering from C. difficile infection, the method comprising orally or enterally administering to the patient an effective amount of a synthetic composition consisting essentially of one or more fucosylated human milk oligosaccharides wherein the synthetic composition is artificially prepared and contains at least one compound that is produced ex vivo chemically and/or biologically.

22. The method of claims 10, wherein the human milk oligosaccharide is administered for at least 14 days.

23. The method of claims 10, wherein the human is administered an amount of 0.5 g to 15 g per day of the one or more human milk oligosaccharides in the synthetic composition.

24. The method of claim 23, wherein the human is first administered a higher dose for a period of time and then administered a lower dose of the human milk oligosaccharide.

25. The method of claim 12, wherein the liver disease or condition is selected from the group consisting of cholesterol gallstones, cirrhosis, non-alcoholic steatohepatitis (NASH), non-alcoholic fatty liver disease (NAFLD) and sclerosing cholangitis.

26. The method of claim 13, wherein the inflammatory bowel disease is Crohn's disease or ulcerative colitis.

27. The method of claim 13, wherein the synthetic composition is administered when the patient is in remission of an inflammatory bowel disease.

28. The method of claim 15, wherein the metabolic disorder is selected from the group consisting of obesity, type II diabetes and syndrome X.

29. The method of claim 19, wherein the condition associated with antibiotic treatment is selected from the group consisting of C. difficile infection, urinary tract infection and antibiotic associated diarrhoea.

30. The method of claim 20, wherein the one or more fucosylated neutral human milk oligosaccharides is selected from the group consisting of 2′-FL, 3-FL, DFL and LNFP-I; and mixtures thereof.

31. The method of claim 20, wherein the one or more non-fucosylated neutral human milk oligosaccharides is LNnT, LNT, or a mixture of LNnT and LNT.

32. The method of claim 22, wherein the human milk oligosaccharide is administered for at least 21 days.

33. The method of claim 23, wherein the human is administered an amount of 1 g to 10 g per day of the one or more human milk oligosaccharides.

34. The method of claim 24, wherein the first higher dose is 3 grams to 10 grams per day and the lower dose is 2 grams to 7.5 grams per day.

35. The method of claim 10, wherein the synthetic composition further contains a bifidobacteria.

36. The method of claim 35, wherein bifidobacterial is selected from the group consisting of Bifidobacterium longum, Bifidobacterium infantis and Bifidobacterium bifidum; or a mixture thereof.
